(19) United States
(12) Patent Application Publication
(10) Pub. No.: US 2009/0214513 A1
(43) Pub. Date: Aug. 27, 2009

(54) MAMMALIAN EXPRESSION SYSTEMS

(75) Inventors: Xiaotian Zhong, Wayland, MA (US); Ronald William Kriz, Northborough, MA (US); Mark Lloyd Stahl, Lexington, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/911,908

(22) PCT Filed: Apr. 20, 2006
(86) PCT No.: PCT/US2006/014859

§ 371 Date: Apr. 22, 2009

Related U.S. Application Data
(60) Provisional application No. 60/672,997, filed on Apr. 20, 2005.

(51) Int. Cl. A61K 38/46; C12N 5/06; C12N 15/87; C12N 5/08; C12P 21/00; A61K 38/02

(52) U.S. Cl. 424/94.67; 435/383; 435/455; 435/366; 435/69.1; 435/69.4; 435/69.6; 435/69.51; 435/69.52; 514/2

(57) ABSTRACT

The present invention features mammalian expression systems with improved production yields, and method of using these systems to produce desired proteins. In one embodiment, the expression systems of the present invention comprise genetically-engineered mammalian host cells cultured in a medium that contains an effective amount of heparin or heparin-like molecules. The presence of heparin or heparin-like molecules significantly increases protein production by the cultured cells. The present invention also features the use of constitutively-active components of FGFR-I-mediated signal transduction pathways to improve protein production by cultured mammalian cells. Co-expression of such a component with a protein of interest markedly increases the production yield of the protein of interest.

### TECHNICAL FIELD

This invention relates to mammalian expression systems and methods of using the same for producing desired proteins.

### BACKGROUND

With recent advances in genomics and proteomics, the ability to clone and express recombinant proteins in large amounts has become increasingly important. The ability to purify high levels of proteins is important in the human pharmaceutics and biotechnology setting, for production of protein pharmaceuticals such as insulin, as well as in the basic research setting, for example to crystallize a protein to allow determination of its three-dimensional structure. Proteins that are otherwise difficult to obtain in quantity can be overexpressed in a host cell and subsequently isolated and purified.

Bacterial expression systems have been one approach to expression and purification of recombinant proteins. However, expression of many eukaryotic polypeptides, and particularly mammalian proteins, in bacterial cells has frequently produced disappointing and unsatisfactory results because conditions and the environment in the host cells were not conducive to correct folding and modification of the eukaryotic protein.

Yeast expression systems offer certain advantages for the production of some eukaryotic proteins, because they have secretory pathways and have the ability to perform some limited post-translational modifications. However, yeast systems often lead to improper folding of disulfide linked proteins, and may result in hypoglycosylation.

The use of mammalian cells for the production of proteins offers the important advantages of providing correct protein folding as well as the appropriate post-translational modifications, such as glycosylation. However, many mammalian expression systems do not produce large quantities of desired proteins.

### SUMMARY OF THE INVENTION

The present invention features the use of heparin, heparin-like molecules, or fibroblast growth factor receptor (FGFR) agonists to increase protein production by mammalian host cells. The present invention also features the use of constitutively-active FGFRs or their downstream effectors to stimulate protein production by mammalian host cells.

In one aspect, the present invention provides mammalian expression systems with improved protein production yields. These expression systems include genetically-engineered mammalian cells cultured in a medium that contains an effective amount of heparin or heparan sulfate glycosaminoglycans. Each of the genetically-engineered host cells includes a recombinant expression cassette encoding a protein of interest. The presence of heparin or heparan sulfate glycosaminoglycans in the culture medium significantly increases the yield of the protein of interest.

The amount of heparin or heparan sulfate glycosaminoglycans used in the present invention can be any amount that is effective for promoting protein production by the cultured host cells. In one embodiment, a culture medium employed in the present invention includes from about 1 to about 1,000 μg/ml of heparin or heparan sulfate glycosaminoglycans. In another embodiment, a culture medium employed in the present invention includes from about 10 to about 200 μg/ml of heparin or heparan sulfate glycosaminoglycans (e.g., about 10, 15, 20, 25, 30, 35, 40, 45, 50, 55, 60, 65, 70, 75, 80, 85, 90, 95, or 100 μg/ml).

In yet another embodiment, a culture medium employed in the present invention is a serum-free medium which includes an effective amount of fibroblast growth factor 2 (FGF-2) or other FGFs, in combination with heparin or heparan sulfate glycosaminoglycans, for increasing protein production by the cultured host cells. In many examples, the culture medium includes, without limitation, from about 10 to about 500 ng/ml of FGF-2 (e.g., about 10, 20, 30, 40, 50, 60, 70, 80, 90, 100, 200, 300, 400, or 500 ng/ml).

In another aspect, the mammalian expression systems of the present invention include genetically-engineered mammalian cells cultured in a medium that contains an effective amount of an FGFR-1 activation agent. Each of these genetically-engineered cells includes a recombinant expression cassette encoding a protein of interest. The presence of the FGFR-1 activation agent in the culture medium markedly increases the yield of the protein of interest. Examples of FGFR-1 activation agents suitable for the present invention include, but are not limited to, FGFs, heparins, heparan sulfate glycosaminoglycans, or other heparin-like molecules. Agents capable of activating other FGFRs can also be used. In one embodiment, the FGFR-1 activation agent employed in the present invention includes both heparin or heparan sulfate glycosaminoglycans and FGF-2.

In still another aspect, the mammalian expression systems of the present invention include genetically-engineered mammalian cells, each of which includes one or more recombinant expression cassettes that encode a protein of interest and a constitutively-active component of an FGFR-1-mediated signal transduction pathway. In one example, the constitutively-active component of the FGFR-1-mediated signal transduction pathway is a constitutively-active FGFR-1 protein.

The present invention also features the use of β-xylosides or other glycosaminoglycan biosynthesis inducers to improve protein production by mammalian cells. Non-limiting examples of β-xylosides suitable for this purpose include 4-methylumbelliferyl-β-D-xyloside, p-nitrophenyl-β-D-xyloside, and benzyl-β-D-xyloside. In one example, mammalian host cells are cultured in a medium that comprises from about 50 or about 100 μg/ml of 4-methylumbelliferyl-β-D-xyloside. The use of β-xylosides significantly increases the protein production yield of mammalian host cells.

Any protein of interest can be produced using the mammalian expression systems of the present invention. Non-limiting examples of these proteins include insulins, growth hormones, growth factors, erythropoietin proteins, follicle-stimulating hormones, interferons, interleukins, cytokines, colony stimulating factors, coagulation factors, tissue plasminogen activators, parathyroid hormones, bone morphogenetic proteins, keratinocyte growth factors, granulocyte colony-stimulating factors, granulocyte-macrophage colony-stimulating factors, glucagons, thrombins, thrombopoietins, protein C, secreted frizzled-related proteins, selectins, antibodies, or viral proteins. These proteins can be secreted, cytosolic, or membrane-bound proteins. They can be used for therapeutic, prophylactic, diagnostic, or other medical purposes. In many examples, the proteins produced by the present invention are incapable of interacting with cell-surface heparan sulfate proteoglycans to induce cellular internalization of these proteins.

The present invention further features pharmaceutical compositions including proteins produced by the mammalian expression systems of the present invention.

In addition, the present invention features methods for producing desired proteins. In one aspect, the methods of the present invention include culturing mammalian host cells in a medium, wherein each of the host cells includes a recombinant expression cassette encoding a protein of interest, and the culture medium contains an effective amount of heparin, heparan sulfate glycosaminoglycans, or an FGFR-1 activation agent for increasing the production of the protein of interest by the host cells; and isolating the protein of interest from the host cells or the culture medium.

In one embodiment, the recombinant expression cassette is carried by an expression vector which is transiently introduced into the host cells. The heparin or heparan sulfate glycosaminoglycans are added to the culture medium at least 24 hours after the expression vector is transiently introduced into the host cells. In another embodiment, the heparin or heparan sulfate glycosaminoglycans are added to the culture medium at least 48 hours after the expression vector is transiently introduced into the host cells.

In another aspect, the methods of the present invention include culturing mammalian host cells in a medium, wherein each of the host cells includes one or more recombinant expression cassettes that encode a protein of interest and a constitutively-active component of an FGFR-1-mediated signal transduction pathway; expressing the protein of interest and the component in the host cells; and isolating the protein of interest from the host cells or the culture medium.

In one embodiment, the constitutively-active component is a constitutively-active FGFR-1 protein. In many instances, the proteins produced using the methods of the present invention are incapable of interacting with cell-surface heparan sulfate proteoglycans to induce cellular internalization of these proteins.

Other features, objects, and advantages of the present invention are apparent in the detailed description that follows. It should be understood, however, that the detailed description, while indicating preferred embodiments of the invention, is given by way of illustration only, not limitation. Various changes and modifications within the scope of the invention will become apparent to those skilled in the art from the detailed description.

### BRIEF DESCRIPTION OF THE DRAWINGS

The drawings are provided for illustration, not limitation.

FIG. 1 depicts the restriction map of an expression vector used in the present invention.

FIG. 2 illustrates the enhancement effect of heparin on protein production in cultured HEK293 cells.

FIG. 3 demonstrates that the optimal concentration of heparin for increasing protein production in cultured HEK293-EBNA cells is about 25 μg/ml.

FIG. 4 shows that heparin increases the production of secreted frizzled-related protein 1 (sFRP-1) in stable HEK293 cell lines.

FIG. 5 is a Northern blot illustrating that heparin does not increase sFRP-1 mRNA levels.

FIG. 6 is a Western blot demonstrating the stimulatory effect of heparin on intracellular protein synthesis.

FIG. 7 demonstrates that purified sFRP-1 without heparin is active and stable. Panel A is a pair of protein elution profiles from conditioned media from transfected 293 cells after a Nickel NTA column. The nickel-purified materials were further purified using size exclusion column Superdex 200. Panel B is a Coomassie blue stained gel of purified sFRP-1-his6. Protein samples after Nickel NTA or Superdex 200 were analyzed by SDS-PAGE under reduced (lanes 1 & 3) or non-reduced conditions (lanes 2 & 4). Panel C represents luciferase assays for Wnt-3 antagonistic activity of purified sFRP-1 using U2OS cells transfected with TCF-luciferase.

FIG. 8 is a Western blot demonstrating that heparin stimulates sFRP-1 production in glycosylation-deficient CHO cell line Lec.3.2.8.1. sFRP-1 transfected Lec.3.2.8.1 cells were mock-treated or treated with 50 μg/ml of heparin 48 h after DNA transfection. Conditioned media were collected at different time points and analyzed by immunoblotting with anti-his4 antibody.

FIG. 9 is a Western blot illustrating effects of modified heparins. sFRP-1-transfected 293 cells were left untreated (−) or were incubated for 72 h with heparin, N-desulfated, N-acetylated heparin (dN-heparin), or 2-O-desulfated heparin (dO-heparin), all at 50 μg/ml. The cells were also treated with 4-methylumbelliferyl 7-β-D-xyloside (Xyloside) at the indicated concentrations. Medium samples were collected and analyzed for immunoblotting with anti-his4 antibody.

FIG. 10 demonstrates that fibroblast growth factor-2 (FGF-2) significantly improves the action of heparin in serum-free media.

FIG. 11 shows that blocking fibroblast growth factor receptor-1 (FGFR-1) markedly reduces the protein production enhancement effect of heparin.

FIG. 12 is a Western blot demonstrating that heparin enhances production of human matrix metalloproteinase 23 (MMP-23) in HEK293 transient expression.

FIG. 13 is a Western blot demonstrating that heparin enhances production of human Dickkopf-1 (DKK-1) in HEK293 transient expression.

### DETAILED DESCRIPTION

The present invention features mammalian expression systems with improved production yields for secreted, cytosolic, or membrane-bound proteins. In one aspect, the expression systems of the present invention comprise genetically-engineered mammalian host cells cultured in a medium that contains an effective amount of heparin or heparin-like molecules. Each genetically-engineered mammalian host cell includes a recombinant expression cassette encoding a protein of interest. The presence of heparin or heparin-like molecules in the culture medium significantly increases the yield of the protein of interest. In another aspect, the expression systems of the present invention employ genetically-engineered mammalian host cells that comprise one or more recombinant expression cassettes encoding a protein of interest and a constitutively-active FGFR-1 or FGFR-1 effector. Co-expression with the FGFR-1 or its effector significantly improves the yield of the protein of interest.

Various aspects of the present invention are described in further detail in the following subsections. The use of subsections is not meant to limit the invention. Each subsection may apply to any aspect of the invention.

### A. USE OF HEPARIN TO INCREASE PROTEIN PRODUCTION BY GENETICALLY-ENGINEERED MAMMALIAN HOST CELLS

Heparin can be used to enhance protein production by genetically-engineered mammalian host cells. Heparin is a heterogeneous mixture of sulfated glycosaminoglycans. The main sugar units in heparin include α-L-iduronic acid 2-sulfate, 2-deoxy-2-sulfamino-α-D-glucose 6-sulfate, α-D-glucuronic acid, 2-acetamido-2-deoxy-α-D-glucose, and α-L-iduronic acid. These sugars are joined by glycosidic linkages, forming polymers of varying sizes. Many heparin molecules include a large amount of disaccharide unit IdoA(2-OSO₃)-GlcNSO₃(6-OSO₃), leading to a heavily O-sulfated polysaccharide with a high iduronic (IdoA) to glucuronic acid (GlcA) ratio. Because of its highly acidic sulfate groups, heparin typically exits as an anion at physiologic pH.

The present invention contemplates the use of any type of heparin, including but not limited to, unfractionated heparin, fractionated heparin, or low-molecular-weight heparin (LMWH). The molecular weight of un-fractionated heparin can range, without limitation, from about 3,000 to about 40,000 Da, with a mean molecular weight of about 15,000 Da (approximately 40-50 monosaccharide units). The average molecular weight of many commercial heparin preparations is in the range of from about 12,000 to about 15,000 Da. Un-fractionated heparin can be prepared from a variety of tissues of vertebrates, such as porcine intestinal mucosa, bovine intestinal tissue, or bovine lung tissue. The preparation process typically involves a proteolytic treatment of the tissue followed by extraction and complexing with ion pairing reagents. Un-fractionated crude heparin can be further subjected to fractional precipitation, purification, or chemical treatment to produce cell-culture-grade or pharmaceutically-acceptable heparin.

LMWH is typically made from un-fractionated heparin by chemical or enzymatic hydrolysis. The molecular weight of many commercial LMWH preparations can range, for example, from about 2,000 to 9,000 Da, with a mean molecular weight of about 4,000 to 5,000 Da.

Without limiting the present invention to any one theory or mode of action, heparin mediates the interaction between FGF (e.g., FGF-2) and FGFR (e.g., FGFR-1), thereby activating or facilitating the activation of FGFR and setting in motion a cascade of downstream signals that leads to increased protein synthesis and/or secretion. Heparin alone may also activate FGFR.

In addition, heparin can bind to other growth factors, cytokines, or chemokines. Non-limiting examples of these growth factors, cytokines, or chemokines include platelet-derived growth factor (PDGF), vascular endothelial growth factor (VEGF), pleiotrophin, placental growth factor (P1GF), platelet factor-4 (PF-4), heparin-binding EGF-like growth factor interleukin-8 (IL-8), hepatocyte growth factor (HGF), macrophage inflammatory protein-1 (MIP-1), transforming growth factor-beta (TGF-beta), interferon-g-inducible protein-10 (IP-10), interferon-gamma (IFN-gamma), and HIV-Tat transactivating factor. Interactions of heparin with these factors or their receptors may also promote protein synthesis by cultured host cells.

The structural requirements of heparin responsible for its interaction with FGF and FGFR have been investigated using different experimental models. The results indicate that size and degree of sulfation are important for the capacity of heparin to induce FGF-2-FGFR interaction. See, e.g., Ishihara et al. (1993) J. Biol. Chem. 268:4675-4683; Tyrrell et al. (1993) J. Biol. Chem. 268:4684-4689; Guimond et al. (1993) J. Biol. Chem. 268:23906-23914; Avezier et al. (1994) J. Biol. Chem. 269:114-121; and Walker et al. (1994) J. Biol. Chem. 269:931-935. The minimal FGF-2-binding sequence in heparin or heparan sulfate has been identified as a pentasaccharide which contains the disaccharide units IdoA(2-OSO₃)— GlcNSO₃ or IdoA(2-OSO₃)-GlcNSO₃(6-OSO₃). See, e.g., Maccarana et al. (1993) J. Biol. Chem. 268:23898-23905. Highly sulfated octa or decasaccharide fragments derived from heparin have also been shown to mediate the interaction between FGFs and their receptors. See, e.g., Klagsbrun et al. (1991) Cell 67:229-231 and Ishihara et al., supra. In addition, heparin-derived tetrasaccharides to octasaccharides have been shown to bind to FGF-2 but be less potent than heparin-derived deca- and longer oligosaccharides in stimulating cell proliferation. See Delehedde et al. (2002) Biochem. J. 365:235-244. Binding studies involving chemically modified heparin or heparan sulfate preparations indicate that 2-O- and N-sulfate groups are important for heparin/heparan sulfate interaction with FGF-2. In addition, it is believed that heparin requires both 2-O- and N-sulfate groups, as well as 6-O-sulfate groups, to promote the binding of FGF-2 to FGFR-1. See, e.g., Guimond et al., supra.

Heparin-binding region(s) on FGF has been tentatively identified in both the NH₂ terminus and COOH terminus of the protein, where basic amino acid residues may interact with sulfate groups of heparin. In addition to promoting the formation of heparin-FGF-FGFR complex, the association with heparin can stabilize FGF and protect it from proteolytic degradation. In addition, FGFs (e.g., FGF-2) can be internalized following direct binding to cell-surface heparan sulfate independently of any interaction with FGFR. This leads to the speculation that HS, in an HS-FGF complex, may serve as a shuttle to transport FGF to the nucleus.

The culture media employed in the present invention can include any amount of heparin that is effective for promoting protein production by the cultured cells. Because of the acidic sulfate groups, heparin salt is typically used for cell cultures. Non-limiting examples of suitable heparin salts include heparin sodium salt, heparin calcium salt, or heparin lithium salt. The concentration of heparin in a culture medium can range, for example, from 1 to 1,000 μg/ml, from 5 to 500 μg/ml, from 10 to 200 μg/ml, from 15 to 100 μg/ml, or from 20 to 30 μg/ml. In one embodiment, the concentration of heparin in a culture medium is about 10, 15, 20, 25, 30, 35, 40, 45, or 50 μg/ml. The heparin employed in the present invention can be of any type, such as un-fractionated heparin, fractionated heparin, or LMWH.

Many types of culture media can be used for the present invention. In one embodiment, a culture medium employed in the present invention includes a base medium supplemented with fetal bovine serum and an effective amount of heparin or heparin-like molecules (e.g., heparan sulfate glycosaminoglycans). In many examples, the culture medium includes at least 0.5%, 1%, 5%, or 10% fetal bovine serum. Other animal sera or tissue extracts can also be used. Examples of suitable base media include, but are not limited to, MEM, MEM-alpha, DMEM, RPMI, ISCOVE, Ham F12, HAM F10, M199, L15, 6M, IMEM, RPMI-1640, NCTC109, Fischer medium, Waymouth medium, Williams medium, Madin-Darby bovine kidney media, Madin-Darby canine kidney media, or mixtures thereof. These base media can be enriched according to the needs of the host cells, with additional nutrient factors such as, for example, sugars (e.g., glucose), amino acids (e.g., glutamine), a cocktail of nonessential amino acids, a cocktail of essential amino acids, peptides, acid salts (e.g., sodium pyruvate), EDTA salts, citric acid derivatives, alcohols (e.g., ethanol), amino alcohols (e.g., ethanolamine), vitamins (e.g., vitamin C or vitamin E), antioxidants (e.g., glutathione or selenium), fatty acids with saturated or unsaturated chains (e.g., linoleic acid, arachidonic acid, oleic acid, stearic acid, or palmitic acid), lipids, lipopeptides, or phospholipids (e.g., lecithins). Buffer solutions, such as those based on HEPES or bicarbonates, can be used for certain fragile cell cultures or for cultures producing large amounts of CO₂. In many cases, care is taken to ensure that the pH of a culture medium remains optimal for cell growth or protein production (e.g., between 6 and 8, between 7 and 8, or between 7.2 and 7.5) and that the culture medium remains isotonic.

The present invention also features the use of serum-free or chemically-defined media. In one embodiment, a serum-free medium employed in the present invention include an effective amount of heparin or heparin-like molecules and an effective amount of FGF(s). The concentration of FGF(s) employed can range, for example, from 1 to 1,000 ng, from 10 to 100 ng, or from 25 to 75 ng. The FGF family includes at least 23 distinct members. These proteins possess broad mitogenic and cell survival activities and are involved in a variety of biological processes including embryonic development, cell growth, morphogenesis, tissue repair, tumor growth and invasion. Sequence homology among different FGF members of the same species is relative low. However, there is considerable species cross-reactivity for FGFs.

In one embodiment, FGF-2 is used in combination with heparin or heparin-like molecules to stimulate protein production by mammalian host cells. In one example, the concentration of heparin or heparin-like molecules employed ranges from 5 to 200 μg/ml (e.g., about 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μg/ml), and the concentration of FGF-2 employed ranges from 10 to 500 ng/ml (e.g., about 10, 20, 30, 40, 50, 60, 70, 80, 90, 100, 200, 300, 400, or 500 ng/ml). FGF-2 has been reported to bind to a variety of FGF receptors, including but not limited to, FGFR-1, FGFR-2, FGFR-3, FGFR-4 and FGFR-5. It has also been reported that heparin or heparin-like molecules are not absolutely required for FGF-2 signaling but that these molecules facilitate signaling at a much lower FGF-2 concentration than is possible in their absence. See Padera et al., FASEB J., 13:1677-1687 (1999).

The present invention further contemplates the use of biologically-active fragments or variants of FGF-2 to promote protein production by mammalian host cells. These biologically active fragments or variants retain at least a substantial portion of the protein production enhancement activity of the original FGF-2 protein. These fragments or variants can be naturally occurring, or deliberately engineered. A desired FGF-2 fragment or variant can be readily prepared by amino acid substitutions, deletions, insertions, or other modifications. The protein production enhancement activity of such a fragment or variant, when used in combination with heparin or heparin-like molecules, can be easily assessed using the methods described in the Examples set forth below.

Mammalian host cells that suitable for the present invention include, but are not limited to, cells that are deficient in heparan sulfate glycosaminoglycan (HSGAG) synthesis, or cells that have reduced levels of cell-surface HSGAGs. Non-limiting examples of suitable mammalian host cells include HEK293-FT (Invitrogen R700-07), HEK293-EBNA (Invitrogen R62007), CHO pgsA-745 (American Type Culture Collection or ATCC), CHO pgsB-650 (ATCC), CHO pgsD-677 (ATCC), CHO pgsB-618 (ATCC), and other heparan sulfate-deficient CHO mutant cells such as those described in Lidholt et al., PROC. NATL. ACAD. SCI. U.S.A., 89:2267-2271 (1992), which is incorporated herein by reference in its entirety. Other mammalian host cells can also be used, such as baby hamster kidney (BHK) cells, HeLa cells, COS-1 cells, myeloma NSO cells, HKB cells, CV-1 cells, C127 cells, Vero cells, Sp-2 cells, Madin-Darby kidney cells, Madin-Darby canine kidney cells, and other cell lines available from ATCC or other commercial sources. In addition, the present invention contemplates the use of primary cell cultures, tissue cultures, organ cultures, or transgenic mammals for the production of a protein of interest. Heparin or heparin-like molecules can be delivered to a transgenic mammal via intravenous infusion, subcutaneous injection, or other suitable routes. In one example, heparin or heparin-like molecules are delivered to a specific tissue site using an implant or catheter.

In addition, the present invention features the use of hybrid or fusion cells for the production of a protein of interest. A hybrid cell can be created by fusing a mammalian cell and a cancer/immortal cell (e.g., a myeloma or blastoma cell). Methods suitable for this purpose include, but are not limited to, electrofusion and chemical fusion (e.g., polyethylene glycol fusion). The mammalian cell and the cancer/immortal cell can be derived from the same or different species. In many embodiments, the cancer/immortal cells are sensitive to one or more selective agents. For instance, the cancer/immortal cells can be sensitive to a culture medium containing hypoxanthine, aminopterin and thymidine, which is known as “HAT medium.” The HAT-sensitive cancer/immortal cells can be fused to mammalian cells that are insensitive to HAT medium. Hybrid cells are selected against HAT, which kills unfused cells. The fused cells can then be screened for desired features.

In one embodiment, a hybrid cell employed in the present invention is a hybridoma cell which produces a monoclonal antibody of interest. Culturing the hybridoma cell in a medium that contains an effective amount of heparin or heparin-like molecules significantly increases the yield of the monoclonal antibody. In another embodiment, a hybrid cell employed in the present invention comprises a recombinant expression cassette which encodes a protein of interest. The recombinant expression cassette can be introduced into the hybrid cell before or after the fusion event. In many cases, the mammalian cells or the cancer/immortal cells used for the preparation of hybrid cells are deficient in HSGAG synthesis or have reduced levels of cell-surface HSGAGs.

Each mammalian host cell employed in the present invention (including hybrid cells) comprises a recombinant expression cassette that encodes a protein of interest. The recombinant expression cassette typically includes a protein coding sequence operatively linked to expression control sequences. The protein coding sequence, which encodes the protein of interest, can be of any type, such as genomic sequence, cDNA, or a combination thereof. Selection of suitable expression control sequences for directing the expression of a protein of interest is a matter of routine design within the level of ordinary skill in the art. Non-limiting examples of suitable expression control sequence include promoters, enhancers, the Kozak sequences, polyadenylation sequences, or other transcription/translation regulatory sequences. Many of these sequences are described in the literature and are available through commercial suppliers. The promoter(s) employed in a recombinant expression cassette can be constitutive or inducible.

A recombinant expression cassette can be introduced into a mammalian host cell by a variety of means. In one embodiment, an expression vector comprising the recombinant expression cassette is introduced into mammalian host cells by transfection or transduction. Exemplary transfection techniques include, but are not limited to, calcium phosphate-mediated transfection, DEAE-dextran-mediated transfection, cationic lipid-mediated, and electroporation. Transduction is typically mediated by recombinant viral vectors. Non-limiting examples of viral vectors suitable for this purpose include retroviral, lentiviral, adenoviral, adeno-associated viral, herpes viral, alphaviral, astrovirus, coronavirus, orthomyxovirus, papovavirus, paramyxovirus, parvovirus, picornavirus, poxvirus, or togavirus vectors. Liposomally-encapsulated expression vectors can also be used for introducing a recombinant expression cassette into mammalian host cells. An expression vector can be either transiently or stably introduced into host cells. In many cases, the expression vector employed includes a selectable marker which allows for the selection of host cells that are transfected or transduced with the vector. Non-limiting examples of suitable selectable markers include neomycin (G418), hygromycin, puromycin, zeocin, colchine, methotrexate, and methionine sulfoximine.

A recombinant expression cassette can also be incorporated into a host cell by modifying an endogenous gene of the cell. The endogenous gene encodes a protein of interest. Any portion of the endogenous gene can be modified to achieve a desired expression or regulation effect. For instance, the promoter of an endogenous gene can be replaced with a viral promoter to increase the level of expression of the gene in the host cell.

Any protein of interest can be produced according to the present invention. Non-limiting examples of these proteins include therapeutic, prophylactic, or diagnostic proteins, such as hormones, growth factors, interleukins, cytokines, interferons, colony stimulating factors, blood factors, antibodies, vaccines, collagens, fibrinogens, human serum albumins, tissue plasminogen activators, anti-coagulants, or replacement enzymes for congenital diseases. Specific examples of these proteins include, but are not limited to, insulin, human growth hormone, erythropoietin, human follicle-stimulating hormone, chorionic gonadotropin, luteinizing hormone, bone morphogenetic protein 2, parathyroid hormone, alpha interferons, beta interferons, gamma interferons, interleukin-1, interleukin-1 antagonists, interleukin-2, interleukin-10, interleukin-11, interleukin-12, keratinocyte growth factor, keratinocyte growth factor-2, human granulocyte colony-stimulating factor, human granulocyte-macrophage colony-stimulating factor, nesiritide, anti-thrombin III, coagulation factor IX, coagulation factor VIII, coagulation factor VIIa, streptokinase, urokinase, glucocerebrosidase, alpha-D-galactosidase, alpha L-iduronidase, alpha-1,4-glucosidase, arylsulfatase B, iduronate-2-sulfatase, adenosine deaminase, deoxyribonuclease, alteplase, myelin basic protein, hypoxanthine guanine phosphoribosyl transferase, tyrosine hydroxylase, dopadecarboxylase, glucagon, monoclonal antibodies targeting leukocyte receptors (e.g., alpha 4 integrin antagonists, anti-thymocyte globulin, CD2 antagonists, CD3 antagonists, CD4 antagonists, CD20 antagonists, CD22 antagonists, CD33 antagonists, and CD52 antagonists), monoclonal antibodies targeting cytokines (e.g., chemokine antagonists, IL-2 antagonists, IL-4 antagonists, IL-5 antagonists, IL-6 antagonists, IL-12 antagonists, selectin antagonists, and TNF-alpha antagonists), monoclonal antibodies targeting cancer cell markers or receptors (e.g., epithelial growth factor antagonists, human epidermal growth factor receptor 2 antagonists, MUC-1 antagonists, and vascular endothelial growth factor antagonists), and other monoclonal antibodies (e.g., complement antagonists, C5 inhibitors, glycoprotein IIb/IIIa antagonists, IgE antagonists, and respiratory syncytial virus F-protein antagonists).

Other types of antibodies or antibody fragment can also be produced according to the present invention. Examples of these antibodies or antibody fragments include, but are not limited to, humanized antibodies, human antibodies, single-chain antibodies, chimeric antibodies, synthetic antibodies, recombinant antibodies, hybrid antibodies, mono-specific antibodies, poly-specific antibodies, non-specific antibodies, Fab fragments, F(ab′)2 fragments, Fv, scFv, Fd, or dAb. High-affinity binders selected by using in vitro display technologies or evolutionary strategies can also be produced according to the present invention. These high-affinity binders include, but are not limited to, peptides, antibodies, or antibody mimics, such as those described in Binz et al. (2004) Nat. Biotechnol. 22:575-582 or Lipovsek et al. (2004) J. Immunol. Methods 290:51-67.

Other proteins of interest that can be produced according to the present invention include, but are not limited to, kinases, phosphatases, G protein coupled receptors, growth factor receptors, cytokine receptors, chemokine receptors, cell-surface antibodies (membrane bound immunoglobulin), BMP/GDF-receptors, neuronal receptors, ion channels, proteases, transcription factors, polymerases, prothrombin, thrombin, alpha-1 antitrypsin, alglucerase, imiglucerase, thrombopoietin, alpha-1 proteinase inhibitor, calcitonin, elcatonin, goserelin, nafarelin, buserelin, pro-insulin, insulin analogues, amylin, C-peptide, somatostatin, octreotride, vasopressin, insulinotrophin, human protein C, cystic fibrosis transmembrane conductance regulator, insulin-like growth factors, nerve growth factors, secreted frizzled-related proteins (e.g., sFRP-1 to 5), or selectins (e.g., selectin P, selectin E, or selectin L).

The present invention also features the production of viral proteins or immunogenic fragments thereof. These viral proteins or fragments can be used to prepare vaccines for eliciting immunoprotective reactions against the corresponding viruses. Non-limiting examples of these viral proteins include proteins of human immunodeficiency viruses (e.g., HIV-1 and HIV-2), influenza viruses (e.g., influenza A, B and C viruses), coronaviruses (e.g., human respiratory coronavirus), hepatitis viruses (e.g., hepatitis viruses A to G), or herpesviruses (e.g., HSV 1-9). Proteins of other viruses can also be produced. These viruses include, but are not limited to, pneumovirus, morbillivirus, rubulavirus, adenovirus, arenavirus, lymphocytic choriomeningitis virus, phlebovirus, hantavirus, torovirus, Ebola-like virus, hepacivirus, flavivirus, simplexvirus, varicellovirus, cytomegalovirus, roseolovirus, lymphocryptovirus, thogotovirus, orthopoxvirus, avipoxvirus, leporipoxvirus, lentivirus, spumavirus, lyssavirus, novirhabdovirus, vesiculovirus, alphavirus, bubivirus, rhinovirus, aphtovirus, poliomyelitisvirus, pseudorabies virus, bovine herpes virus, paramyxovirus, newcastle disease virus, respiratory syncitio virus, mumps virus, measles virus, a parvovirus, papovavirus, rotavirus, gastroenteritisvirus, tick-borne encephalitis virus, yellow fever virus, rubella virus, hog cholera virus, or rabies virus.

A protein of interest produced by the present invention can be, without limitation, a secreted protein, a cytosolic protein, or a membrane-bound protein. The sequence of a protein of interest can be either naturally-occurring or genetically-engineered. In one embodiment, a protein of interest is a fusion protein comprising a polypeptide tag which facilitates the isolation, purification, detection, immobilization, stabilization, folding, or targeting of the protein of interest. Non-limiting examples of suitable polypeptide tags include streptavidin tags, FLAG tags, c-myc tags, poly-histidine tags, influenza HA tags, VSV glycoprotein tags, V5 tags, herpes simplex virus tags, glutathione S-transferase, or Fc fragments. In some cases, the polypeptide tags can be cleaved from the proteins of interest by a selected protease.

In another embodiment, a protein of interest comprises a signal peptide which facilitates the secretion of the protein by the mammalian host cells. The signal peptide can be either endogenous or heterologous to the protein of interest. A signal peptide can interact with signal recognition particles and direct ribosomes to the ER where co-translational insertion takes place. Many signal peptides are highly hydrophobic with positively charged residues. A signal sequence can be removed from the growing peptide chain by a signal peptidase, a protease located on the cisternal face of the ER. Therefore, in many cases, a secreted protein isolated from the culture medium does not have the original signal peptide.

In many embodiments, a secreted or membrane-bound protein produced by the present invention does not bind to or interact with cell-surface HSGAGs. This prevents or reduces the uptake or internalization of the protein by the mammalian host cells. In one example, a protein of interest produced by the present invention is not a heparanase or an enzyme whose substrate is heparan sulfate (HS).

The proteins of interest produced by the present invention can be isolated or purified by a variety of means. Non-limiting examples of initial materials that are suitable for protein isolation/purification include culture media or cell lysates. Exemplary isolation methods include, but are not limited to, affinity chromatography (including immunoaffinity chromatography), ionic exchange chromatography, hydrophobic interaction chromatography, size-exclusion chromatography, HPLC, protein precipitation (including immunoprecipitation), differential solubilization, electrophoresis, centrifugation, crystallization, or combinations thereof.

In many embodiments, an isolated protein of interest is substantially free from other proteins or contaminants. For instance, the isolated protein of interest can be at least 50%, 60%, 70%, 80%, 90%, 95%, 99% or 100% pure from other proteins. In one example, an isolated protein contains no more than an insignificant amount of contaminants that would interfere with its intended uses. A protein of interest isolated according to the present invention can be verified or evaluated using standard techniques such as SDS-PAGE or immunoassays. Immunoassays suitable for this purpose include, but are not limited to, Western blots, ELISAs, RIAs, sandwich or immunometric assays, latex or other particle agglutination, or proteomic chips. Protein sequencing and mass spectroscopy can also be used to verify or analyze an isolated protein of interest.

Furthermore, the present invention contemplates the use of heparin or heparin-like molecules to increase the production of attenuated viruses by mammalian host cells. These attenuated viruses can be used for the preparation of vaccine formulations. Suitable mammalian host cells for this purpose include, but are not limited to, CHO cells, BHK cells, Vero cells, Madin-Darby kidney cells, or Madin-Darby canine kidney cells. The amount of heparin or heparin-like molecules employed for this purpose can be any amount that is effective in improving the yield of the attenuated viruses (e.g., from about 10 to 1,000 μg/ml, such as about 10, 15, 20, 25, 30, 35, 40, 45, 50, 55, 60, 65, 70, 75, 80, 85, 90, 95, or 100 μg/ml). Where the culture medium is a serum-free medium, FGF-2 or other FGFR agonists can also be used in combination with heparin or heparin-like molecules. The effective amount of FGF-2 or other FGFs can range, without limitation, from 10 to 500 ng/ml (e.g., about 10, 20, 30, 40, 50, 60, 70, 80, 90, 100, 200, 300, 400, or 500 ng/ml). In addition, FGFs can be added to a culture medium that contains an animal serum to further improve the yield of the attenuated viruses or other proteins of interest.

### B. USE OF HEPARIN-LIKE MOLECULES OR OTHER FGFR AGONISTS TO INCREASE PROTEIN PRODUCTION BY GENETICALLY-ENGINEERED MAMMALIAN HOST CELLS

The present invention also features the use of heparin-like molecules or other FGFR agonists to stimulate protein production by mammalian host cells. Non-limiting examples of heparin-like molecules include sulfated glycosaminoglycans (GAGs), such as heparan sulfate glycosaminoglycans (HSGAGs); sulfated proteoglycans, such as heparan sulfate proteoglycans (HSPGs); or fragments thereof. Other heparin-like molecules that are suitable for the present invention include, but are not limited to, heparin oligosaccharides, synthetic sulfated polymers, various sulfated molecules, various sulfonated molecules, synthetic polyaromatic compounds, polyaromatic compounds synthesized by polymerization of aromatic ring, or combinations or fragments thereof. Heparin-like molecules, as described in Casu (1985) Advances in Carbohydrate Chemistry and Biochemistry 43:51-134, which is incorporated herein by reference, can also be used. All of these heparin-like molecules can stimulate protein production by mammalian host cells (e.g., heparan sulfate-deficient mammalian host cells). In many cases, the heparin-like molecules employed in the present invention are highly sulfated and can facilitate the activation of FGFR signaling in the cultured cells.

At least four distinct FGFR family members have been identified—namely, FGFR-1, FGFR-2, FGFR-3, and FGFR-4. FGFRs differ from one another in their ligand affinities and tissue distribution. A full-length representative FGFR includes an extracellular region, composed of multiple immunoglobulin-like domains, a single hydrophobic membrane-spanning segment, and a cytoplasmic tyrosine kinase domain. In one embodiment, the heparin-like molecules employed in the present invention can activate or facilitate the activation of FGFR-1 in the cultured cells.

The activation of cell-surface FGFRs is believed to involve the interactions among FGF, FGFR, and cell-surface HSGAGs. At least three distinct models have been proposed to explain how FGF and cell-surface HSGAG co-operate to induce the activation of FGFR. In the “growth hormone” model, a single FGF dimerizes two FGFRs, with HSGAG binding to both FGF and the extracellular domain of the FGFR. In the “HS-dependent dimerization” model, the HS chain binds to two FGFs and the now dimeric ligand dimerizes the FGFR. In the “dimer-of-dimers” model, two independent complexes of FGF and HSGAG cause the dimerization of the FGFR, thereby activating its intracellular kinase domain.

The cell-surface GAG chains are often attached to a small protein core to form HSPGs. HSPGs can be anchored to cell surfaces through a hydrophobic transmembrane domain of the core protein or through a glycosyl-phosphatidylinositol (GPI) anchor covalently bound to the core protein (transmembrane HSPGs). Non-limiting examples of transmembrane HSPGs include, but are nor limited to, glypican, cerebroglycan, betaglycan, perlecan, CD44, and various members of the syndecan family such as syndecan 1, syndecan 2 (fibroglycan), syndecan 3 (N-syndecan) and syndecan 4 (ryudocan). Glypican and cerebroglycan can also be coupled to cell membranes through covalent GPI anchors. In addition, HSPGs can be non-covalently attached to cell surfaces through interactions with cell-surface macromolecules (peripheral membrane HSPGs).

In one embodiment, the present invention features the use of soluble HSGAGs or HSPGs, or fragments thereof, to enhance protein production by cultured mammalian cells. Soluble HSGAGs or HSPGs, or their fragments, can be prepared by chemical or enzymatic digestion of immobilized or larger HSGAG or HSPG molecules. For instance, transmembrane or GPI-anchored HSPGs can be released from cell membranes by proteolytic digestion of their core protein or action of endogenous phospholipases. Likewise, HSGAG chains or fragments can be prepared by chemical or enzymatic digestion of the polysaccharidic backbone. An effective amount of soluble HSGAGs or HSPGs (or their fragments) can be added to a culture medium to stimulate protein production by the cultured cells. In many instances, the amount of soluble HSGAGs or HSPGs (or their fragments) employed is equivalent to about 10-200 μg/ml of heparin (e.g., about 10, 20, 30, 40, 50, 60, 70, 80, 90 or 100 μg/ml of heparin) for the protein production enhancement effect created.

In one example, the soluble HSGAGs or HSPGs (or fragments thereof) employed in the present invention comprise at least one disaccharide unit IdoA(2-OSO₃)-GlcNSO₃ or IdoA(2-OSO₃)-GlcNSO₃(6-OSO₃). In another example, the soluble HSGAGs or HSPGs (fragments thereof) employed in the present invention comprise a penta, octa or decasaccharide which contains the disaccharide unit IdoA(2-OSO₃)-GlcNSO₃ or IdoA(2-OSO₃)-GlcNSO₃(6-OSO₃). In still another example, the soluble HSGAGs or HSPGs (or fragments thereof) employed in the present invention comprise a plurality of disaccharide units IdoA(2-OSO₃)-GlcNSO₃ or IdoA(2-OSO₃)-GlcNSO₃(6-OSO₃).

In addition to soluble forms, heparin or heparin-like molecules can also be immobilized on a substrate to stimulate protein production by mammalian host cells. In one example, the substrate provides physical support for the cultured host cells (e.g., a heparin- or heparin-like molecule-coated culture plate).

Moreover, protein production by mammalian host cells can be increased by enhancing the endogenous synthesis of HSGAGs or HSPGs in the cells. The backbone of the heparan sulfate chain appears to be synthesized by heparan sulfate synthase, which possesses both glucuronosyltransferase and N-acetylglucosaminyltransferase activities. The backbone can be further modified by a series of sulfotransferases and carbohydrate-modifying enzymes, including N-deacetylase/N-sulfotransferase, C-5 epimerase, 2-O sulfotransferase, 6-O sulfotransferase, and 3-O sulfotransferase. Therefore, by increasing the expression or activities of these enzymes, the cell-surface HSGAGs or HSPGs can be increased, resulting in improved protein production by the cells. To achieve this, expression vectors encoding the above enzymes can be introduced into mammalian host cells and co-expressed with a protein of interest.

### C. CO-EXPRESSION OF CONSTITUTIVELY-ACTIVE FGFRS OR FGFR EFFECTORS TO INCREASE PROTEIN PRODUCTION BY GENETICALLY-ENGINEERED MAMMALIAN HOST CELLS

Constitutively-active FGFRs or FGFR down-stream effectors can be used to promote protein production by cultured mammalian host cells. Constitutively-active FGFRs generated via mutation, gene fusion or other genetic alternations have been observed in many human cancers. Examples of constitutively-active FGFR mutants include, but are not limited to, FGFRs with the thanatophoric dysplasia type I mutation (e.g., Arg250→Cys mutation in FGFR1 or SEQ ID NO:1, and Arg248→Cys mutation in FGFR3 or SEQ ID NO:2), and FGFRs with a missense mutation in the activation loop of the kinase domain (e.g., Lys656→Glu mutation in FGFR1 or SEQ ID NO:1, and Lys650→Glu mutation in FGFR3 or SEQ ID NO:2). Other constitutively-active FGFR mutants, such as those described in De Moerlooze et al. (1997) Curr. Opin. Genet. Dev. 7:378-385, Wang et al. (2002) Cancer Res. 62:1898-1903, or Wang et al. (2004) Prostate 58:1-12, all of which are incorporated herein by reference, can also be used. In addition, the present invention contemplates the use of constitutively-active chimeric FGFRs, such as that described in Kudla et al. (1998) J. Cell Biol. 142:241-250, which is incorporated herein by reference. Recombinant expression cassettes encoding these constitutively-active FGFRs can be readily constructed and introduced into mammalian host cells. Co-expression with such a constitutively-active FGFR can significantly increase the yield of the protein of interest.

The present invention also features the use of FGFR downstream effectors to improve protein production by mammalian host cells. Non-limiting examples of FGFR effectors include Crk (v-crk sarcoma virus CT10 oncogene homolog), phospholipase C gamma, fibroblast growth factor receptor substrate 2, and SHC (Src homology 2 domain containing) transforming protein. All of these proteins are SH₂-domain proteins, as they can bind to phosphotyrosine residues of FGFR during its activation. Other FGFR effectors that can be used in the present invention include various components in the MAPK signaling pathway, such as GRB2 (growth factor receptor-bound protein 2), SOS1 (son of sevenless homolog 1), RRAS2 (related RAS viral (r-ras) oncogene homolog 2), RAF1 (v-raf-1 murine leukemia viral oncogene homolog 1), MAP2K1 (mitogen-activated protein kinase kinase 1), MAP2K2 (mitogen-activated protein kinase kinase 2), MAPK1 (mitogen-activated protein kinase 1), SRF (serum response factor or c-fos serum response element-binding transcription factor), FOS (v-fos FBJ murine osteosarcoma viral oncogene homolog), ELK1 (ELK1, member of ETS oncogene family), ELK4 (ELK4, ETS-domain protein, or SRF accessory protein 1), and c-MYC (v-myc myelocytomatosis viral oncogene homolog). Like constitutively-active FGFRs, recombinant expression cassettes encoding FGFR downstream effectors can be easily constructed and introduced into mammalian host cells and co-expressed with a protein of interest.

### D. GLYCOSAMINOGLYCAN BIOSYNTHESIS INDUCERS

The present invention further features the use of β-xylosides or other glycosaminoglycan (GAG) biosynthesis inducers to improve protein production by mammalian cells. Heparan sulfate is synthesized in vivo as a glycosaminoglycan component of heparan sulfate proteoglycans. It has been shown that GAG biosynthesis is initiated by the transfer of a xylose residue from UDP-Xyl to the hydroxyl group of a serine residue on the core protein, followed by the addition of two Gal residues and one GlcA residue to form a linkage tetrasaccharide GlcAβ1-3Glaβ1-3Galβ1-4Xylβ1. Heparan sulfate is then synthesized on this linkage tetrasaccharide. It has also been reported that addition of a β-xyloside to cell culture medium induces elongation of GAG chains.

The present invention demonstrates that addition of a β-xyloside to a culture medium can significantly increase protein production by the cultured mammalian host cells. Non-limiting examples of suitable β-xylosides include, but are not limited to, 4-methylumbelliferyl-β-D-xyloside, p-nitrophenyl-β-D-xyloside, and benzyl-β-D-xyloside. The concentration of a β-xyloside in a culture medium can range, for example, from 10 to 500 μg/ml, from 20 to 200 μg/ml, or from 50 to 100 μg/ml. The cultured medium can further include an animal serum (e.g., 1%, 5%, or 10% of fetal bovine serum), or be serum-free. Where a serum-free medium is used, FGF-2 or other FGF factors can be supplemented to further improve the production yield of the cultured mammalian host cells. In one example, a culture medium employed in the present invention comprises from about 20 to about 200 μg/ml of 4-methylumbelliferyl-β-D-xyloside. In another example, the culture medium includes from about 50 to about 100 μg/ml of 4-methylumbelliferyl-β-D-xyloside. Any protein of interest described herein can be produced by a mammalian expression system enhanced by β-xylosides or other GAG biosynthesis inducers.

### E. PHARMACEUTICAL COMPOSITIONS

The therapeutic or prophylactic proteins produced by the present invention can be used to prepare pharmaceutical compositions for the treatment or prevention of human disease. A pharmaceutical composition of the present invention typically includes a therapeutically or prophylactically effective amount of a protein of interest and a pharmacologically acceptable carrier. As used herein, “pharmaceutically acceptable carrier” can be any solvent, dispersion medium, coating, antibacterial or antifungal agent, isotonic or absorption delaying agent, or the like. The use of such media and agents for pharmaceutically active substances is well known in the art. Supplementary active ingredients can also be incorporated into a pharmaceutical composition of the present invention.

Administration of a pharmaceutical composition of the present invention can be via any common route so long as the target tissue is available via that route. This includes oral, nasal, buccal, rectal, vaginal, or topical. Alternatively, administration can be by orthotopic, intradermal, subcutaneous, intramuscular, intraperitoneal, intratumoral, circumferentially, catheterization, or intravenous injection.

A pharmaceutical composition of the present invention can also be administered parenterally or intraperitoneally. Solutions of proteins of interest can be prepared in water suitably mixed with a surfactant, such as hydroxypropylcellulose. Dispersions can also be prepared in glycerol, liquid polyethylene glycols, or mixtures thereof, or in oils. Under ordinary conditions of storage and use, these preparations can contain a preservative to prevent the growth of microorganisms.

The pharmaceutical forms suitable for injectable use include sterile aqueous solutions or dispersions, or sterile powders for the extemporaneous preparation of sterile injectable solutions or dispersions. In many cases, the pharmaceutical forms are sterile and fluid to the extent that easy syringability exists. The pharmaceutical forms can also be stable under the conditions of manufacture and storage and preserved against the contaminating action of microorganisms, such as bacteria and fungi. Suitable pharmaceutical carriers include, but are not limited to, solvents or dispersion media containing, for example, water, ethanol, polyol (e.g., glycerol, propylene glycol, and liquid polyethylene glycol, and the like), or vegetable oils. The proper fluidity can be maintained, for example, by the use of a coating, such as lecithin, by the maintenance of the required particle size in the case of dispersion, or by the use of surfactants. The prevention of the action of microorganisms can be brought about by various antibacterial or antifungal agents, for example, parabens, chlorobutanol, phenol, sorbic acid, thimerosal, or the like. In many cases, it will be preferable to include isotonic agents, for example, sugars or sodium chloride. Prolonged absorption of the injectable compositions can be brought about by the use of agents delaying absorption, for example, aluminum monostearate and gelatin.

Sterile injectable solutions can be prepared by incorporating a therapeutic or prophylactic protein in the required amount in an appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients. In the case of sterile powders for the preparation of sterile injectable solutions, the preferred methods of preparation are vacuum-drying or freeze-drying techniques which yield a powder of the active ingredient plus any additional desired ingredient from a previously sterile-filtered solution thereof.

For oral administration, a therapeutic or prophylactic protein produced by the present invention can be incorporated with excipients and used in the form of non-ingestible mouthwashes and dentifrices. A mouthwash can be prepared incorporating a therapeutic or prophylactic protein in the required amount in an appropriate solvent, such as a sodium borate solution (Dobell's Solution). Alternatively, a therapeutic or prophylactic protein can be incorporated into an antiseptic wash containing sodium borate, glycerin and potassium bicarbonate. A therapeutic or prophylactic protein can also be dispersed in dentifrices, gels, pastes, powders, or slurries.

Upon formulation, compositions or solutions can be administered in a manner compatible with the dosage formulation and in such amount as is therapeutically or prophylactically effective. The dosage regimen can be determined by the attending physician based on various factors such as the action of the protein, the site of pathology, the severity of disease, the patient's age, sex and diet, the severity of any inflammation, time of administration, and other clinical factors. In one example, systemic or injectable administration is initiated at a dose which is minimally effective, and the dose is increased over a pre-selected time course until a positive effect is observed. Subsequently, incremental increases in dosage are made limiting to levels that produce a corresponding increase in effect while taking into account any adverse affects that may appear.

Toxicity and therapeutic efficacy of a therapeutic or prophylactic protein can be determined by standard pharmaceutical procedures in cell culture or experimental animal models. For instance, the LD50 (the dose lethal to 50% of the population) and the ED50 (the dose therapeutically effective in 50% of the population) of a protein of interest can be determined using conventional means. The dose ratio between toxic and therapeutic effects is the therapeutic index, and can be expressed as the ratio LD50/ED50. In many cases, therapeutic proteins that exhibit large therapeutic indices are selected.

It should be understood that the above-described embodiments and the following examples are given by way of illustration, not limitation. Various changes and modifications within the scope of the present invention will become apparent to those skilled in the art from the present description.

### F. EXAMPLES

### Example 1

### Cell Culture and DNA Constructs

Mammalian cell lines (HEK293-FT, HEK293-EBNA, CHO-DUKX, and Lec.3.2.8.1) were grown and maintained in a humidified incubator with 5% CO₂ at 37° C. HEK293 cells were cultured in free-style 293 media (Invitrogen) supplemented with 5% fetal bovine serum (FBS). CHO-DUKX stable lines were grown in alpha media containing 10% FBS and 200 mM methotrexate (MTX). HEK293 stable lines were cultured in alpha media containing 10% FBS and 100 nM MTX.

Transient expression was performed in either 50-ml spinners or 1-L spinners. For 50 ml culture volumes, 25 μg of plasmid DNA were mixed with 400 μg of polyethylenimine (PEI, 25 kDa, linear, neutralized to pH 7.0 by HCl, 1 mg/ml (Polysciences, Warrington, Pa.) in 2.5 ml of serum-free 293 media. For 1 liter culture volumes, 500 μg of DNA were mixed with 4 mg of PEI in 50 ml of serum-free 293 media. The mixtures were then mixed with either 50 ml or 1 liter of HEK293 cells in 293 media with 5% FBS at a cell density of 0.5×10⁶ cells/ml in spinners. The spinners were incubated at 37° C. with a rotation rate of 170 rpm on a P2005 Stirrer (Bellco) for 72-144 hours before harvest.

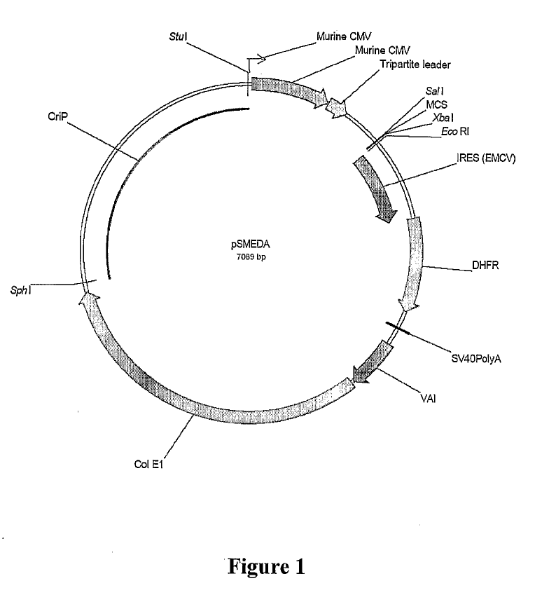

Two vectors (pSMED2 and pSMEDA) were used for the DNA constructs. pSMEDA, a derivative from pSMED2, is described in FIG. 1. An OriP element was inserted into pSMEDA so that the vector can be amplified in cells containing EBNA-1 viral antigen. This vector allows several folds of increase in protein production in HEK293-EBNA cells. For sFRP-1 (secreted frizzled-related protein 1) constructs, a C-terminal His6 tag and the mutation VFK312-314LE were incorporated into PCR primers before a stop codon. The PCR products were digested with SalI and EcoRI. The gel purified DNA fragments were subcloned into pSMED2 (creating pWZ1028), or into pSMEDA (creating pWZ1049).

For the establishment of CHO-DUKX sFRP-1 stable lines, construct pWZ1028 was transfected into CHO-DUKX cells and the transfectomas were selected against 50 nM, 100 nM or 200 mM of methotrexate (MTX) for three weeks. After screening 72 colonies, three clones (named 200-10, 200-11, and 200-12) resistant to 200 nM MTX with the highest expression levels of sFRP-1 were isolated. HEK293 cells were also found to be sensitive to MTX at the concentration of 100 mM and above, even though they have two copies of the dihydrofolate reductase (DHFR) gene. To construct a HEK293 stable line for sFRP-1, pWZ1028 was transfected into HEK293-EBNA and the transfectomas were selected against 100 nM or 250 nM of MTX for three weeks. Two of the best clones, 100-5 and 100-20, were then isolated.

### Example 2

### Heparin Enhances sFRP-1 Production by Transfected Cells

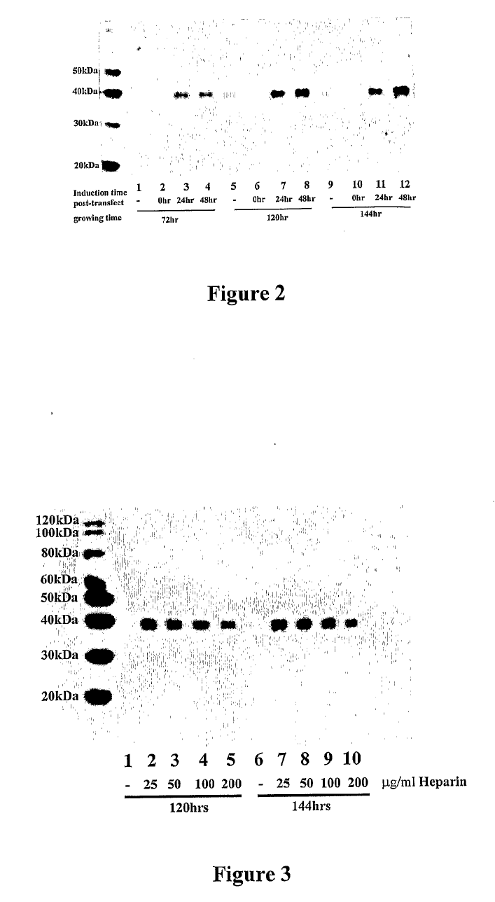

C-terminal his6 tagged sFRP-1 was transiently expressed in HEK293-FT cells as described in Example 1. 50 μg/ml of heparin (Sigma Chemical Co.) was added to the cell culture during or after DNA transfection (“Induction time post-transfection” in FIG. 2). Conditioned media were harvested at different time points (“growing time” in FIG. 2). Protein samples were separated by SDS-PAGE and immunoblotted with mouse monoclonal anti-his4 antibodies (Qiagen) at 0.2 μg/ml (FIG. 2). Immunoblotting was performed as described in Zhong et al. (2004) FEBS Lett. 562:111-117. As illustrated in FIG. 2, heparin significantly increased sFRP-1 production by transfected HEK293 cells. The greatest increase was observed when heparin was added to the culture medium 48 hours after DNA transfection. In other experiments, no such increase was observed for recombinantly-expressed aggrecanase-1 or aggrecanase-2 proteins (data not shown).

C-terminal His6 tagged sFRP-1 was also transiently expressed in HEK293-EBNA cells. Different concentrations of heparin were added to the cell culture 48 hours after DNA transfection (“μg/ml Heparin” in FIG. 3). Conditioned media were harvested at 120 hours or 144 hours after DNA transfection. Protein samples were separated by SDS-PAGE and immunoblotted with mouse monoclonal anti-his4 antibody (FIG. 3). FIG. 3 shows that the optimal heparin concentration for protein production enhancement is about 25 μg/ml.

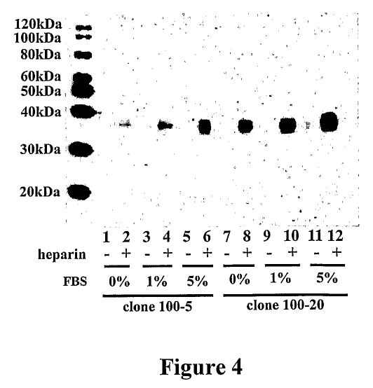

In addition to transiently transfected cells, stable HEK293 cell lines for sFRP-1 (clone 100-5 and 100-20) were also used to evaluate the stimulatory effect of heparin on protein production. Clone 100-5 and 100-20 cells were grown in the presence of different concentrations of fetal bovine serum (“FBS” in FIG. 4). 50 μg/ml of heparin were added with fresh media. Conditioned media were harvested 72 h after heparin treatment. Protein samples were separated by SDS-PAGE and immunostained with mouse monoclonal anti-his4 antibody (FIG. 4). Heparin significantly enhanced sFRP-1 production by clone 100-5 and 100-20 cells. sFRP-1 production also increased with increasing concentration of fetal bovine serum (FBS), indicating that FBS includes factor(s) capable of stimulating protein production in mammalian cells.

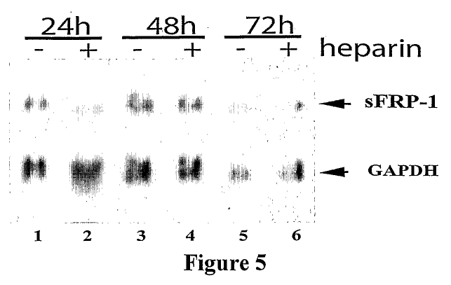

The effect of heparin on mRNA levels was investigated by Northern blot analysis. Total RNA was prepared from 293 cells using RNAqueous (Ambion). 10 μg of RNA was resolved by 1.1% agarose/2% formaldehyde MOPS gel electrophoresis, blotted onto Nytran Supercharge membranes (Schleicher and Schuell) with 8×SSC and hybridized overnight at 50° C. with digoxigenin-labeled DNA probes in DIG easy Hyb solution (Roche). After washing at 60° C. (GAPDH) with 0.5×SSC/0.1% SDS and 0.2×SSC/0.1% SDS, the membranes were blocked in Blocking reagent (Roche) for 30 minutes and probed with alkaline-phosphatase-labeled anti-digoxigenin antibody (Roche) for 30 minutes and with Tris saline buffer/0.3% Tween 20. Signals were visualized with Supersignal (Pierce). Probes were generated by PCR using digoxigenin-labeled nucleotides (Roche). As shown in FIG. 5, heparin does not increase mRNA levels of sFRP-1, as there is no significant difference in mRNA amount between heparin treated and mock-treated cells (lane 1 vs 2, 3 vs 4, 5 vs 6). Therefore heparin does not affect DNA transcription of sFRP-1 or the mRNA stability. It appears to affect post-mRNA processes of sFRP-1 during its protein synthesis.

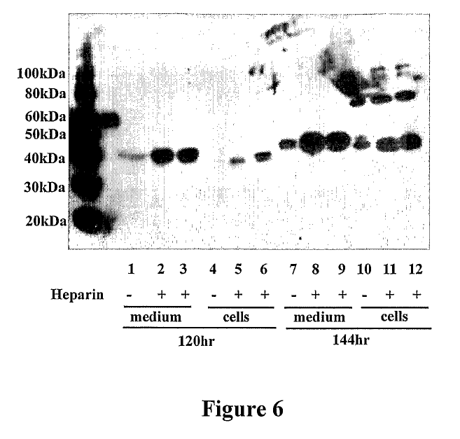

To evaluate the effect of heparin on intracellular protein synthesis, sFRP-1 was transiently expressed in HEK293-FT cells as described above. 50 μg/ml of heparin were added 48 h after DNA transfection. Conditioned media and cell pellets were harvested at 120 h or 144 h. Each protein sample was separated by SDS-PAGE and immunoblotted with mouse monoclonal anti-His4 antibody (FIG. 6). Heparin increased both intracellular and extracellular sFRP-1, suggesting that heparin can enhance mammalian cell protein production by stimulating intracellular protein synthesis. In addition, no cellular uptake of secreted sFRP-1 was observed. This indicates that the accumulation of sFRP-1 in the culture media is not a result of inhibition of HSPG-mediated endocytosis by the heparin added to the media.

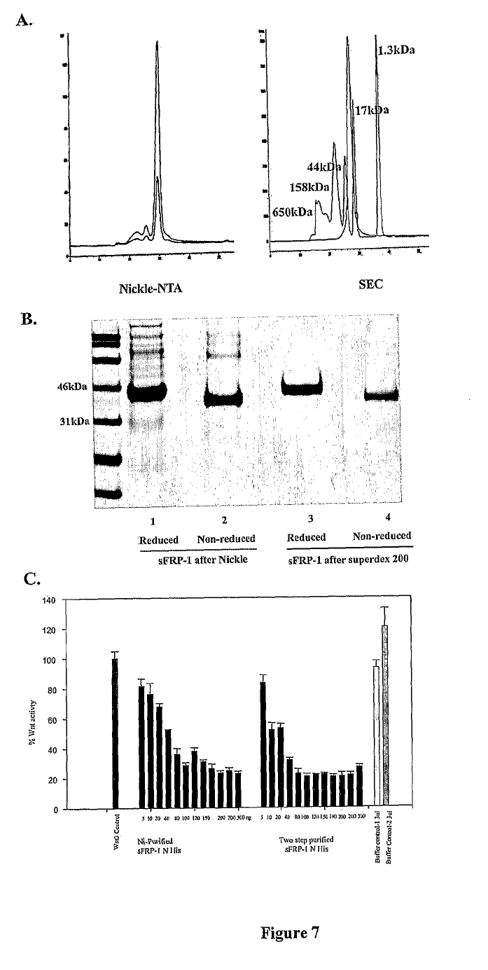

To find out whether the enhancement effect of heparin on sFRP-1 is due to protein stabilization, sFRP-1 produced by HEK293 cells was purified. As described in Example 1, one liter of conditioned medium from transient expression was prepared. The sFRP-1-his6 containing medium was equilibrated with Nickel-NTA (Qiagen) at 4° C. for about an hour. The resin was centrifuged at 3,000 rpm (SORVALL H-6000A/HBB-6) and packed into a column (Pharmacia Biotech) before attaching to HPLC. After an extensive wash with 1M NaCl, 25 mM Tris-HCl pH 7.5, and 15 mM imidazole, sFRP-1-his6 protein was eluted with 1M NaCl, 25 mM Tris-HCl pH 7.5 and 200 mM imidazole. The eluate was concentrated using 10K MWCO concentrators (Vivascience) down to 2 ml. The sample was further passed through a Superdex™ 200 size-exclusion column (SEC) column (Pharmacia Biotech) in 1M NaCl, 25 mM Tris-HCl pH 7.5. The protein was substantially purified after Nickel-NTA (FIG. 7A, left panel and FIG. 7B, lanes 1 & 2), and it was nearly homogeneous after the Superdex 200 (FIG. 7A, right panel and FIG. 7B, lanes 3 & 4).

The protein concentration was determined by absorbance at 280 nm. The protein yield after Nickel-NTA is about 2.5 mg/L and that after SEC is about 1 mg/L. sFRP-1-his6 runs as a monomer in the SEC analysis (FIG. 7A, right panel), which is consistent with sFRP-1 migration as a 38 kDa polypeptide under non-reducing conditions (FIG. 7B, lane 4). N-terminal sequencing and mass spectrometry analysis confirmed the identity of sFRP-1 and showed that the protein was heavily glycosylated (data not shown). When incubated at room temperature, the purified sFRP-1 protein is very stable in the absence of heparin (data not shown). To determine whether the purified sFRP-1 protein was active, the Wnt3 antagonistic activity of sFRP-1 was measured. As shown in FIG. 7C, in the transfected U20S cells, Wnt3 increases the TCF luciferase reporter gene expression (Bhat et al. (2004) Protein Expr. Purif. 37:327-335). The addition of either nickel-NTA purified sFRP-1 or SEC purified sFRP-1 decreased the Wnt-mediated response in a dose-dependent manner, while the buffer had no effect on the Wnt-mediated TCF-luciferase reporter activation. These data clearly demonstrate that, in the absence of heparin, purified sFRP-1 is stable and functional.

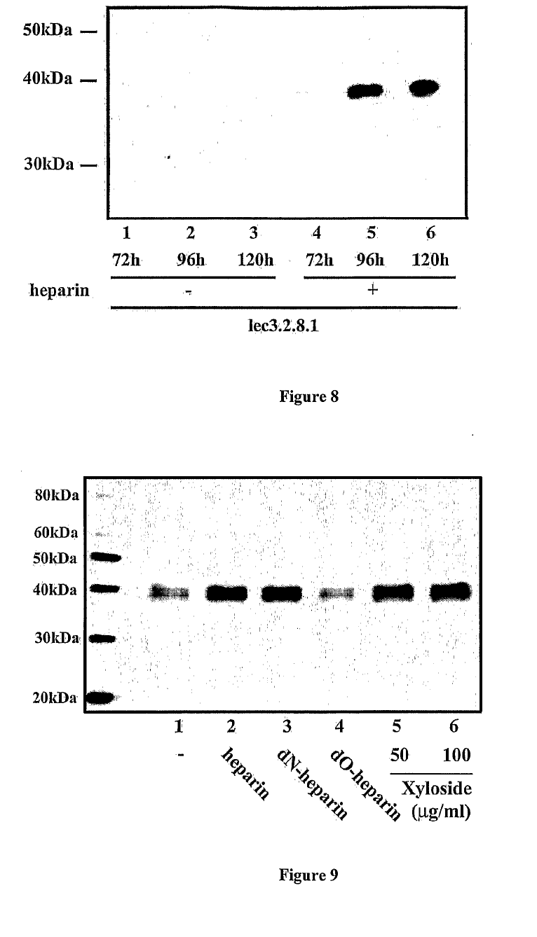

The stimulatory effect of heparin on protein production was also observed in Chinese Hamster Ovary (CHO) cells. Wild-type CHO cells can synthesize endogenous heparin-like molecules, heparin sulfate glycosaminoglycans (HSGAGs). However, when wild-type CHO cells were pretreated with 30 mM chlorate (an inhibitor of heparan sulfate synthesis) for 48 hours, the cells became heparin sensitive. Experiments showed that heparin enhanced protein production in chlorate-treated CHO cells under both transient and stable expression conditions. To further characterize the requirements of heparin for sFRP-1 secretion, the glycosylation deficient CHO cell line Lec3.2.8.1, which carries four glycosylation mutations (Stanley (1989) Mol. Cell. Biol. 9:377-383), was used. This cell line expresses the most drastically modified carbohydrate structures with severely truncated N-linked and O-linked carbohydrates. As shown in FIG. 8, heparin stimulated sFRP-1 secretion in the mutant CHO line (lanes 1-6).

Interaction of heparin-binding proteins with HS is determined by the sequence and sulfation level of the sugar moieties of HS (Esko et al. (2002) Annu. Rev. Biochem. 71:435-471). To test whether O-sulfation and N-sulfation are required for the heparin activity in sFRP-1 secretion, sFRP-1 transfected 293 cells were incubated with chemically modified heparin that was totally N-desulfated, followed by N-acetylation (Sigma Chemical Co.). Modified heparin lacking 2-O-sulfation (Sigma) was also examined for its ability to stimulate sFRP-1 secretion. As shown in FIG. 9, 2-O-desulfated heparin completely lost its ability to stimulate sFRP-1 secretion (lane 4). In contrast, N-desulfated heparin was as efficient as the unmodified heparin (lane 3), suggesting that O-sulfation but not N-sulfation is necessary for the stimulation of sFRP-1. Moreover, the addition of 4-methylumberlliferyl 7-β-D-xyloside (Sigma Chemical Co.), a xyloside that substitutes for the liner moiety to the proteoglycan core protein and thus functions as a soluble primer for glycosaminoglycan biosynthesis, stimulated sFRP-1 secretion at concentrations of 50 and 100 μg/ml (lanes 5 & 6), mimicking the effect of heparin.

### Example 3

### FGF-2 Enhances Protein Production by Transfected Cells

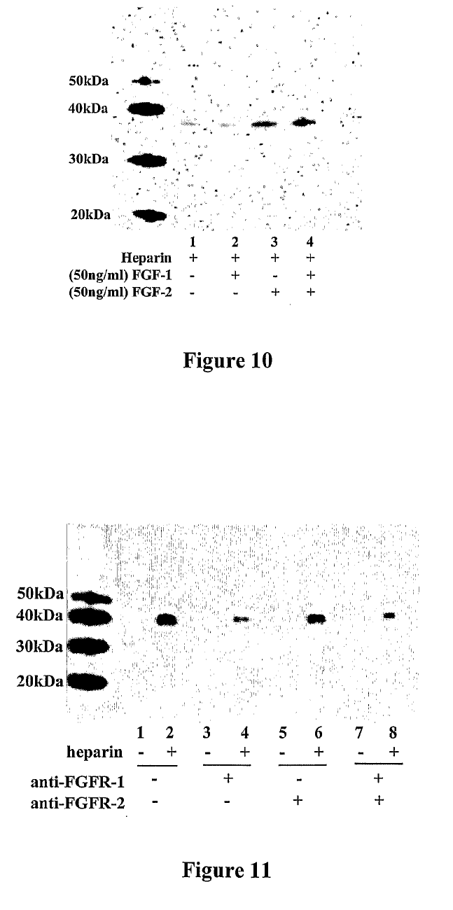

Cells from an HEK293 line stably overexpressing sFRP-1 (clone 100-5) was grown to confluence in a 6-well plate; the media were then replaced with fresh serum-free media containing 50 μg/ml of heparin. 50 ng/ml of fibroblast growth factor-1 (FGF-1, Sigma Chemical Co.) or fibroblast growth factor-2 (FGF-2, Sigma Chemical Co.) were added to the media in corresponding wells. Conditioned media were harvested 48 h after the media replacement. Protein samples were separated by SDS-PAGE and immunoblotted with mouse monoclonal anti-His4 antibody (FIG. 10). As demonstrated in FIG. 10, the combination of FGF-2 and heparin dramatically increased sFRP-1 production by stably transfected HEK293 cells.

Thus, FGF-2 and heparin appear to regulate protein synthesis post-transcriptionally, as Northern analysis showed that the mRNA level of sFRP-1 is not affected by the presence of heparin (FIG. 5). Without wishing to be bound by theory, heparin may affect the processes of protein translation, as FGFs have been shown to influence the translation of their target proteins (Szebenyi et al. (1999) Int. Rev. Cytol.). FGFs may also activate some of the target genes whose products can up-regulate the translation process. Another possibility is that the FGF pathway may facilitate protein trafficking along the secretory pathway. More and more evidence has indicated that protein secretion and surface localization are tightly regulated by a series of signal transduction pathways such as unfolded protein responses (Schroder et al. (2005) Annu. Rev. Biochem. 74:739-789). A number of ER chaperones have been shown to promote cell surface localization and secretion of different client proteins. Heparin and FGF-2 may activate these machineries and facilitate secretion of recombinant proteins.

### Example 4

### Stimulation of FGFR-1 Enhances Protein Production in Transfected Cells

Cells from an HEK293 cell line stably overexpressing sFRP-1 (100-5) were grown to confluence in a 6-well plate and then pre-treated or mock-treated with rabbit polyclonal anti-FGFR-1 or anti-FGFR-2 antibodies (Sigma Chemical Co.) for six hours. The cells were subsequently treated with 50 μg/ml of heparin. Conditioned media were harvested 48 h after the heparin treatment. Protein samples were separated by SDS-PAGE and immunoblotted with mouse monoclonal anti-His4 antibody (FIG. 11). The data indicates that the pretreatment with anti-FGFR-1, not with anti-FGFR-2, significantly reduced the heparin-induced protein enhancement.

### Example 5

### Heparin Enhances MMP-23 and DKK-1 Protein Production in Transfected Cells

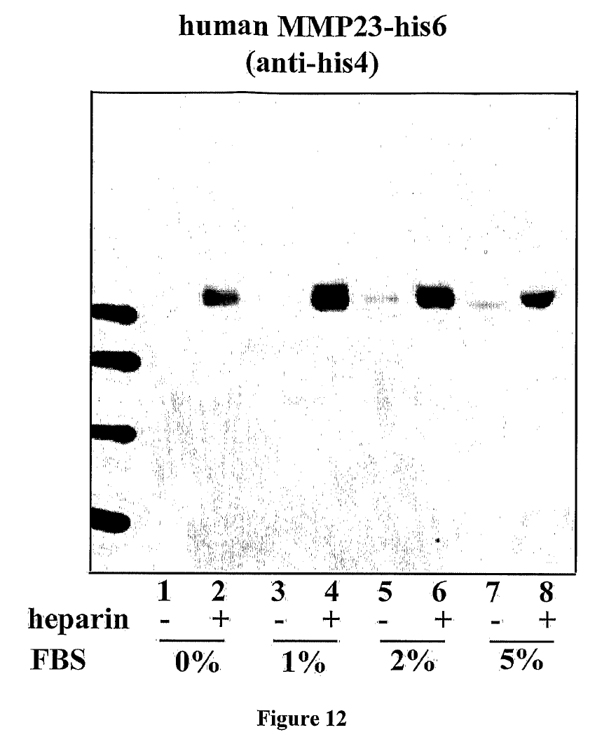

Metalloproteinase MMP-23 with a C-terminal His6 tag in pSMEDA was transiently expressed in HEK293-EBNA cells in medium supplemented with 5% FBS. 24 hours after transfection, the transfected cells were switched to fresh media with various concentrations of FBS. 50 μg/ml of heparin were added to some reactions. Conditioned media were harvested at 96 hours. Protein samples were separated by SDS-PAGE and immunoblotted with mouse monoclonal anti-his4 antibodies. As shown in FIG. 12, heparin significantly increased the observed levels of MMP23-his6 protein.

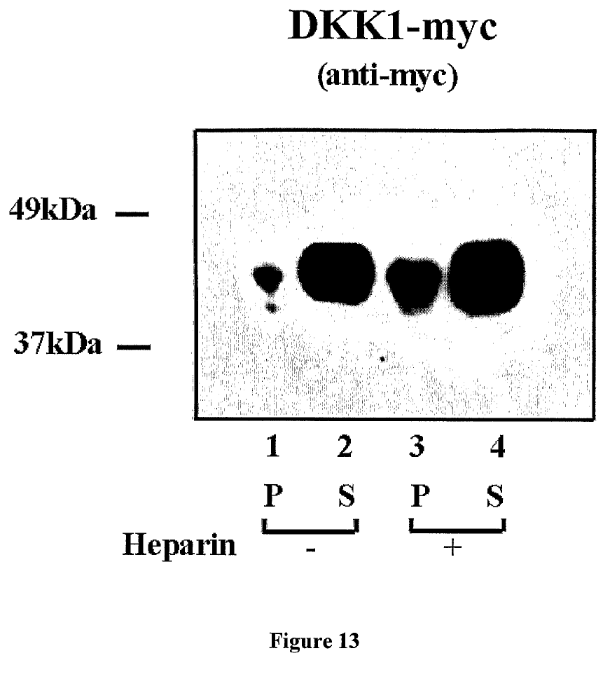

Human Dickkopf-1 (DKK-1) with a C-terminal myc tag in pcDNA3.1 (Invitrogen) was transiently expressed in HEK293-FT cells in medium supplemented with 5% FBS. 50 μg/ml of heparin were added to the cell culture 24 hours after DNA transfection. Conditioned media (S) and cell pellets (P) were harvested at 96 hours. Protein samples were separated by SDS-PAGE and immunoblotted with mouse monoclonal anti-myc antibodies. As shown in FIG. 13, heparin significantly increased the levels of DKK1-myc protein.

The foregoing description of the present invention provides illustration and description, but is not intended to be exhaustive or to limit the invention to the precise one disclosed. Modifications and variations consistent with the above teachings are possible or may be acquired from practice of the invention. Thus, it is noted that the scope of the invention is defined by the claims and their equivalents.

What is claimed is:

1. An expression system comprising mammalian host cells cultured in a medium, wherein each said cell comprises a recombinant expression cassette encoding a protein of interest, and said protein of interest is incapable of interacting with cell-surface heparan sulfate proteoglycans to induce cellular internalization of said protein, and wherein said medium comprises an effective amount of heparan sulfate for increasing the production of said protein by said cells.

2. The expression system of claim 1, wherein said protein of interest is selected from the group consisting of an insulin, a growth hormone, a growth factor, an erythropoietin protein, a follicle-stimulating hormone, an interferon, an interleukin, a cytokine, a colony stimulating factor, a coagulation factor, a tissue plasminogen activator, a parathyroid hormone, a bone morphogenetic protein, a keratinocyte growth factor, a granulocyte colony-stimulating factor, a granulocyte-macrophage colony-stimulating factor, a glucagon, a thrombin, a thrombopoietin, a protein C, a secreted frizzled-related protein, a selectin, a metalloproteinase, a dickkopf protein, and an antibody.

3. The expression system of claim 1, wherein said medium comprises from about 1 to about 1,000 μg/ml of heparan sulfate.

4. The expression system of claim 1, wherein said medium comprises from about 10 to about 200 μg/ml of heparan sulfate.

5. The expression system of claim 1, wherein said medium is a serum-free medium which comprises an effective amount of FGF-2 for increasing the production of said protein by said cells.

6. The expression system of claim 1, wherein said protein of interest is a protein involved in bone development.

7. The expression system of claim 6, wherein said protein of interest is selected from the group consisting of a bone morphogenetic protein, a secreted frizzled-related protein, a metalloproteinase, and a dickkopf protein.

8. A pharmaceutical composition comprising the protein of interest produced by the expression system of claim 1.

9. An expression system comprising genetically-engineered mammalian cells, each of which includes one or more recombinant expression cassettes encoding a protein of interest and a constitutively-active component of an FGFR-1-mediated signal transduction pathway.

10. The expression system of claim 9, wherein said component is a constitutively-active FGFR-1 protein.

11. The expression system of claim 10, wherein said protein of interest is selected from the group consisting of an insulin, a growth hormone, a growth factor, an erythropoietin protein, a follicle-stimulating hormone, an interferon, an interleukin, a cytokine, a colony stimulating factor, a coagulation factor, a tissue plasminogen activator, a parathyroid hormone, a bone morphogenetic protein, a keratinocyte growth factor, a granulocyte colony-stimulating factor, a granulocyte-macrophage colony-stimulating factor, a glucagon, a thrombin, a thrombopoietin, a protein C, a secreted frizzled-related protein, a selectin, a metalloproteinase, a dickkopf protein, and an antibody.

12. The expression system of claim 10, wherein said protein of interest is a protein involved in bone development.

13. The expression system of claim 10, wherein said protein of interest is selected from the group consisting of a bone morphogenetic protein, a secreted frizzled-related protein, a metalloproteinase, and a dickkopf protein.

14. An expression system comprising mammalian host cells cultured in a medium, wherein each said cell comprises a recombinant expression cassette encoding a protein of interest, and said protein of interest is incapable of interacting with cell-surface heparan sulfate proteoglycans to induce cellular internalization of said protein, and wherein said medium comprises an effective amount of an FGFR-1 agonist for increasing the production of said protein by said cells.

15. The expression system of claim 14, wherein said medium is a serum-free medium and comprises heparin or heparin sulfate, and said FGFR-1 agonist comprises
fibroblast growth factor-2.

16. An expression system comprising mammalian host cells cultured in a medium, wherein each said cell comprises a recombinant expression cassette encoding a protein of interest, and said medium comprises an effective amount of β-xyloside for increasing the production of said protein by said cells.

17. The expression system of claim 16, wherein said medium comprises from about 50 μg/ml to about 100 μg/ml of 4-methylumbelliferyl-β-D-xyloside.

18. A method for enhancing expression of a recombinantly-expressed protein, the method comprising the steps of:
culturing mammalian cells in a medium, wherein the mammalian cells comprise a messenger RNA encoding the protein; and
administering to the cells an amount of a FGFR agonist to activate FGFR-1,
wherein the activation of FGFR-1 enhances translation of the protein, and wherein the protein itself is not an activator of FGFR-1.

19. The method of 18, wherein the protein is not a viral protein.

20. The method of claim 18, wherein the medium comprises heparin or heparan sulfate.

21. The method of claim 20, wherein the medium comprises 10-200 μg/ml of heparin.

22. The method of claim 21, wherein each cell includes a transiently-introduced expression vector comprising a recombinant expression cassette for the messenger RNA and the heparin or heparan sulfate is added to the medium at least 24 hours after the expression vector is transiently introduced into the cells.

23. The method of claim 20, wherein the medium is a serum-free medium further comprising an effective amount of FGF-2 for enhancing translation of the protein.

24. The method of claim 20, wherein the medium further comprises serum.

25. The method of claim 18, wherein the medium comprises an effective amount of β-xyloside.

26. The method of claim 25, wherein the medium is a serum-free medium further comprising FGF-2.

27. The method of claim 25, wherein the medium further comprises serum.

28. The method of claim 18, wherein the mammalian cells are human cells.

29. The method of claim 18, wherein the protein is incapable of interacting with cell-surface heparan sulfate proteoglycans to induce cellular internalization of the protein.

30. The method of claim 18, the method further comprising the step of isolating the protein from the cells or the medium.

31. The method of claim 18, where the protein is selected from the group consisting of an insulin, a growth hormone, a growth factor, an erythropoietin protein, a follicle-stimulating hormone, an interferon, an interleukin, a cytokine, a colony stimulating factor, a coagulation factor, a tissue plasminogen activator, a parathyroid hormone, a bone morphogenetic protein, a keratinocyte growth factor, a granulocyte colony-stimulating factor, a granulocyte-macrophage colony-stimulating factor, a glucagon, a thrombin, a thrombopoietin, a protein C, a secreted frizzled-related protein, a selectin, a metalloproteinase, a dickkopf protein, and an antibody.

32. A pharmaceutical composition comprising the recombinantly-expressed protein according to the method of claim 18.

33. A method for producing a protein, comprising:
culturing mammalian cells in a medium, each said cell comprising one or more recombinant expression cassettes encoding said protein and a constitutively-active component of an FGFR-1-mediated signal transduction pathway;
expressing said protein and said component in said cells; and
isolating said protein from said cells or medium.

34. An expression system comprising mammalian host cells cultured in a medium, wherein each said cell comprises a recombinant expression cassette encoding a protein of interest, and said protein of interest is incapable of interacting with cell-surface heparan sulfate proteoglycans to induce cellular internalization of said protein, and wherein said medium comprises an effective amount of heparin for increasing the production of said protein by said cells, wherein the effective amount is (i) greater than 10 μg/ml and less than 100 μg/ml, and (ii) greater than 100 μg/ml and less than or equal to 1,000 μg/ml.

35. The expression system of claim 34, wherein the effective amount is between 15 μg/ml and 75 μg/ml.

36. The expression system of claim 35, wherein the effective amount is 25 μg/ml or 50 μg/ml.

37. The expression system of claim 34, wherein said protein of interest is selected from the group consisting of an insulin, a growth hormone, a growth factor, an erythropoietin protein, a follicle-stimulating hormone, an interferon, an interleukin, a cytokine, a colony stimulating factor, a coagulation factor, a tissue plasminogen activator, a parathyroid hormone, a bone morphogenetic protein, a keratinocyte growth factor, a granulocyte colony-stimulating factor, a granulocyte-macrophage colony-stimulating factor, a glucagon, a thrombin, a thrombopoietin, a protein C, a secreted frizzled-related protein, a selectin, a metalloproteinase, a dickkopf protein, and an antibody.
The expression system of claim 1, wherein said medium is a serum-free medium which comprises an effective amount of FGF-2 for increasing the production of said protein by said cells.

38. The expression system of claim 34, wherein said protein of interest is a protein involved in bone development.

39. The expression system of claim 38, wherein said protein of interest is selected from the group consisting of a bone morphogenetic protein, a secreted frizzled-related protein, a metalloproteinase, and a dickkopf protein.

40. A pharmaceutical composition comprising the protein of interest produced by the expression system of claim 34.

41. A method of treating a disease, comprising administering a therapeutically effective amount of a protein to a patient, (a) wherein the protein is produced according to a method comprising the steps of (i) culturing mammalian cells comprising a messenger RNA encoding the protein, and (ii) activating FGFR-1; (b) wherein activation of FGFR-1 enhances translation of the recombinantly-expressed protein; and (c) wherein the protein itself is not capable of activating FGFR-1.

42. The method according to claim 41, wherein the protein is a matrix metalloproteinase 23 (“MMP23”).

43. The method according to claim 41, wherein the protein is a dickkopf-1 protein (“DKK-1”).

44. The method according to claim 41, wherein the protein is a frizzled-related protein-1 (“FRP-1”).

45. A method of providing protection against a disease, comprising administering a prophylactically effective amount of a protein to a patient, (a) wherein the protein is produced according to a method comprising the steps of (i) culturing mammalian cells comprising a messenger RNA encoding the protein, and (ii) activating FGFR-1; (b) wherein activation of FGFR-1 enhances translation of the recombinantly-expressed protein; and (c) wherein the protein itself is not capable of activating FGFR-1.

46. The method according to claim 45, wherein the protein is a matrix metalloproteinase 23 (“MMP23”).

47. The method according to claim 46, wherein the protein is a dickkopf-1 protein (“DKK-1”).

48. The method according to claim 47, wherein the protein is a frizzled-related protein-1 (“FRP-1”).

49. A method for producing a protein, comprising (a) culturing mammalian cells in a medium, each said cell comprising one or more recombinant expression cassettes encoding said protein, and the medium comprising heparin or heparan sulfate, wherein the heparin is at a concentration of either (i) greater than 10 μg/ml and less than 100 μg/ml, or (ii) greater than 100 μg/ml and less than or equal to 1,000 μg/ml; (b) expressing said protein and said component in said cells; and (c) isolating said protein from said cells or medium.

50. The method according to claim 49, wherein the heparan sulfate is at a concentration of from about 5 μg/ml to about 1,000 μg/ml.
